(19) United States
(12) Patent Application Publication
(10) Pub. No.: US 2006/0188553 A1
(43) Pub. Date: Aug. 24, 2006

(54) Facial cosmetic devices

(76) Inventor: Cathy Meriaux, Lewisburg, PA (US)

Correspondence Address: LAW OFFICES OF ERIC KARICH, 2807 ST. MARK DR., MANSFIELD, TX, 76063, US

(21) Appl. No.: 11/407,594

(22) Filed: Apr. 20, 2006

Related U.S. Application Data
(63) Continuation-in-part of application No. 10/998,203, filed on Nov. 26, 2004.
(60) Provisional application No. 60/587,649, filed on Jul. 14, 2004.
(60) Provisional application No. 60/673,115, filed on Apr. 20, 2005.

(51) Int. Cl. A61K 9/70

(52) U.S. Cl. 424/443

(57) ABSTRACT

An eyeliner element adapted to be applied to a lower portion of an upper eyelid, and an under eye concealer element adapted to be applied to an upper portion of a lower eyelid each include a thin film substrate, an adhesive layer, and a coloring layer formed opposite the adhesive layer.

### CROSS-REFERENCE TO RELATED APPLICATIONS

This application for a utility patent is a continuation-in-part of a previously filed utility patent, still pending, having the application Ser. No. 10/998,203, filed Nov. 26, 2004. This application also claims the benefit of U.S. Provisional Application No. 60/587,649, filed Jul. 14, 2004, and U.S. Provisional Application No. 60/673,115, filed Apr. 20, 2005. These previous applications are hereby incorporated by reference in their entirety.

### STATEMENT REGARDING FEDERALLY SPONSORED RESEARCH

Not Applicable

### BACKGROUND OF THE INVENTION

1. Field of the Invention

This invention relates generally to cosmetic devices, and more particularly to a cosmetic device that is applied to the face in sheet form with an adhesive.

2. Description of Related Art

In general, the term “cosmetic” generally refers to something applied to the body (e.g., the skin or hair) to beautify or to improve a deficiency, defect, or irregularity. In particular, the term generally applies to a preparation, such as powder or a skin cream, designed to beautify the body by direct application. The term “makeup” generally refers to cosmetics that are applied to the face.

It's human nature to want to be considered physically attractive. Evidence of the use of eye makeup has been found in Egyptian tombs dating to 3500 B.C. Today women of all ages around the world wear makeup to enhance their physical appearance. Common cosmetics include various creams, lotions, powders, and other agents for protecting and moisturize the skin, coloring or shadowing the skin, and otherwise covering or concealing any form of blemishes or imperfections.

The use of creams, lotions, and powders have several drawbacks. First, they can be difficult to apply for maximum benefit, and professional application is expensive. Second, they can be troublesome to remove. Third, due to their fluid nature, they are not completely effective in covering or concealing wrinkles, creases, or blemishes that involve the contour or texture of the skin.

It would be beneficial to have a cosmetic device that can be applied to the skin of a person's face in the form of a printed film, which can be later removed without damage to the skin. Such cosmetic devices and systems may be used to cover up an imperfect or damaged facial feature, or even to replace a missing facial feature.

### SUMMARY OF THE INVENTION

The present invention teaches certain benefits in construction and use which give rise to the objectives described below.

The present invention provides an eyeliner element adapted to be applied to a lower portion of an upper eyelid, and an under eye concealer element adapted to be applied to an upper portion of a lower eyelid. The eyeliner element and the under eye concealer element each include a thin film substrate, an adhesive layer, and a coloring layer formed opposite the adhesive layer.

A primary objective of the present invention is to provide an eyeliner element and a under eye concealer element each having advantages not taught by the prior art.

Another objective is to provide a cosmetic product in a thin film substrate form that can easily be applied and removed without specialized training or skill.

A further objective is to provide a cosmetic product that appear identical to professionally applied fluid cosmetics.

Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIG. 1 is a perspective view of one embodiment of a cosmetic device, wherein the cosmetic device is an eye shadow element;

FIG. 2 is a cross sectional view of one embodiment of the eye shadow element of FIG. 1 as indicated in FIG. 1;

FIG. 3 is an exploded perspective view of a cosmetic system including a second embodiment of the cosmetic device of FIG. 1 and a release sheet, wherein the cosmetic device includes a pair of lip-shaped elements;

FIG. 4 is a cross sectional view of the cosmetic system of FIG. 3 as indicated in FIG. 3, wherein the lip-shaped elements are releasably attached to the release sheet;

FIG. 5 is a perspective view of a third embodiment of the cosmetic device of FIG. 1, wherein the cosmetic device is a facial sheet adapted to be applied to a person's face;

FIG. 6 is a view of a cosmetic system including two copies of the facial sheet of FIG. 5 releasably mounted to a release sheet and a container containing a concealer;

FIG. 7 is a perspective view of another embodiment of the cosmetic device of FIG. 1, wherein the cosmetic device is an eyebrow element adapted to resemble a human eyebrow;

FIG. 8 is a cross sectional view of one embodiment of the eyebrow element of FIG. 7 as indicated in FIG. 7;

FIG. 9 is a perspective view of an eyeliner element applied to a lower portion of an upper eyelid just above an upper eyelash;

FIG. 10 is a cross sectional view of one embodiment of the eyeliner element of FIG. 9;

FIG. 11 is a view of one embodiment of a cosmetic system including various embodiments of pairs of the eyeliner element of FIG. 9, an eyeliner pencil, an eye shadow system, and a mascara system;

FIG. 12 is a perspective view of an eye during application of components of the cosmetic system of FIG. 11;

FIG. 13 is a view of one embodiment of a cosmetic system including two cosmetic systems and the cosmetic system of FIGS. 3 and 4;

FIG. 14 is a perspective view of an eye during application of components of the cosmetic systems of FIGS. 11 and 13;

FIG. 15 is a perspective view of an eye wherein an under eye concealer element has been applied to the lower eyelid, and wherein the under eye concealer element extends below the lower eyelid and over a portion of an adjacent cheek, and wherein the under eye concealer element includes several different colors forming a visual pattern or graphic;

FIG. 16 is a perspective view of an eye wherein an under eye concealer element has been applied to the lower eyelid, and wherein the under eye concealer element extends below the lower eyelid and over a portion of an adjacent cheek, and wherein the under eye concealer element forms an image, and has a perimeter that substantially conforms to an outer edge of the image;

FIG. 17 is a perspective view of a portion of a face including an eye and an underlying cheek, wherein the eyeliner element of the cosmetic system of FIG. 11 has been applied to the lower portion of the upper eyelid just above the upper eyelash, the applicator of the eye shadow system of the cosmetic system is being used to apply the eye shadow to the upper eyelid, and a sticker has been applied to the cheek;

FIG. 18 is a perspective view of a portion of a face including an eye and an underlying cheek, wherein an eye shadow element of the cosmetic system of FIG. 13 has been applied to the upper portion of the upper eyelid, and a sticker has been applied to the cheek.

### DETAILED DESCRIPTION OF THE INVENTION

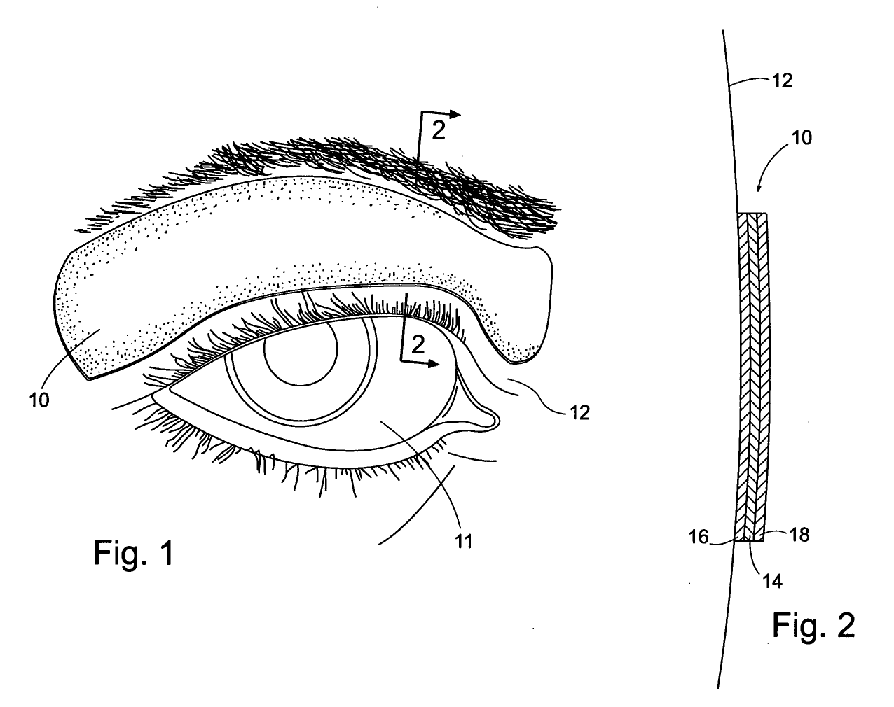

FIG. 1 is a perspective view of one embodiment of a cosmetic device 10, wherein the cosmetic device 10 is an eye shadow element 12. The eye shadow element 12 is shaped to fit over (i.e., substantially cover) an upper portion 14 of an upper eyelid 16 of a person's face 18, and to give the appearance of professionally applied eye shadow. In human eye surface anatomy, an upper eyelid crease 20 separates the upper portion 14 of the upper eyelid 16 from a lower portion 22. As shown in FIG. 1, the upper portion 14 of the upper eyelid 16 generally extends from the upper eyelid crease 20 to an eyebrow 24. When the person blinks, the lower portion 22 of the upper eyelid 16 spreads tears over an outer surface of an eyeball 26, while the upper portion 14 moves very little.

FIG. 2 is a cross sectional view of one embodiment of the eye shadow element 12 of FIG. 1 as indicated in FIG. 1. In the embodiment of FIG. 2, the eye shadow element 12 is a laminate structure including a thin film substrate 30 with an adhesive layer 32 formed on one side and a coloring layer 34 formed on an opposite side. In the embodiment of FIG. 2, the substrate 30 is formed from a thin, flexible material that is not readily visible on the skin. The substrate 30 may be formed from, for example, a thin plastic film material that is substantially transparent. Suitable plastic materials for the substrate 30 are believed to include polyethylene, polypropylene, polyvinyl chloride, and polyurethane.

The adhesive layer 32 is preferably a layer of a pressure sensitive adhesive material that adheres to skin and can be removed from the skin without damaging the skin. The adhesive material is preferably non-toxic and non-irritating. Suitable pressure sensitive adhesive material are well known in the medical and cosmetic arts, and are believed to include acrylic adhesives, rubber based adhesives, and styrene adhesives. The adhesive layer 32 may also be a layer of a water based and/or water washable adhesive material.

To reduce skin irritation, the substrate 30 preferably has many small holes passing therethrough (i.e., is preferably porous), and the adhesive layer 32 preferably conforms to the porous pattern of the substrate 30 such that the underlying skin can “breathe.” For example, the substrate 30 may be formed from a thin film material having many small holes (i.e., from a “microporous” thin film material).

In general, the coloring layer 34 includes coloring agent that simulates either a makeup color or a human skin color. The coloring agent may be, for example, a suitable paint or a stain. The coloring agent is preferably water resistant. The coloring layer 34 preferably has an outer surface that reflects light in substantially the same way that human skin reflects light. The coloring agent may be, for example, sprayed (e.g., airbrushed) onto the substrate 30.

The substrate 30 may be formed from a film material that already includes the adhesive layer 32. Alternately, the substrate 30 may be formed from a material that is inherently adhesive such that the adhesive layer 32 is unnecessary and may be eliminated. For example, GLAD® Press 'n Seal™ sealable plastic wrap (The Glad Products Company, Oakland, Calif.) is a thin plastic film that either includes an adhesive layer or is inherently adhesive, and may be a suitable material from which to form the substrate 30.

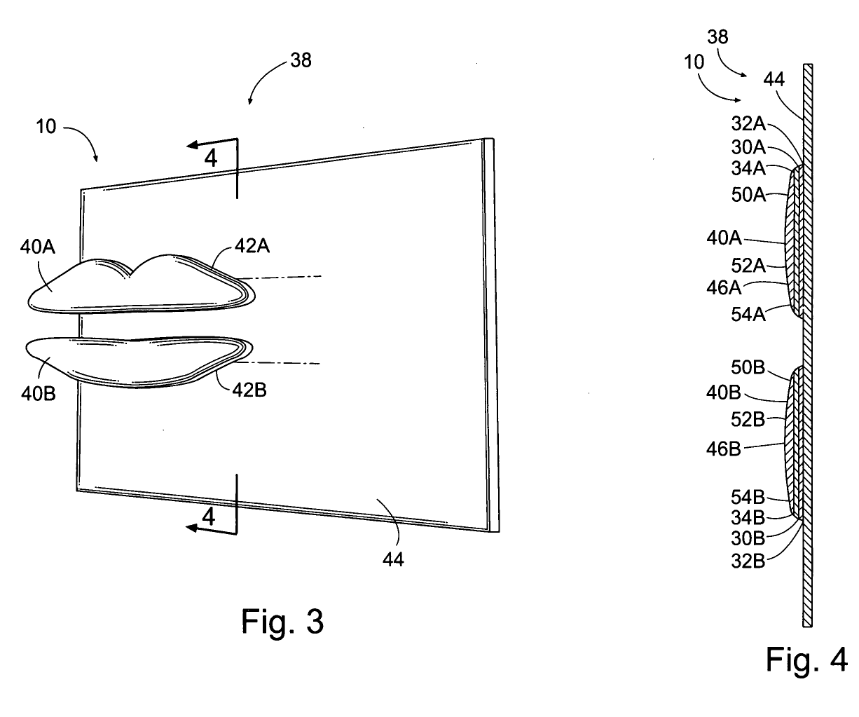

FIG. 3 is an exploded perspective view of a cosmetic system 38 including a second embodiment of the cosmetic device 10 of FIG. 1 and a release sheet 44, wherein the cosmetic device 10 includes a pair of lip-shaped elements 40A and 40B. In general, the lip-shaped elements 40A and 40B may be used to enhance a person's lips.

The lip-shaped elements 40A and 40B are shaped to fit over (i.e., substantially cover) a person's lips. More specifically, the lip-shaped element 40A has a perimeter 42A that is adapted to approximate the person's upper lip, and the lip-shaped element 40B has a perimeter 42B that is adapted to approximate the person's lower lip. Coloring layers of the lip-shaped elements 40A and 40B preferably include a coloring agent that simulates a lipstick color, but may also include a coloring agent that simulates a human skin color.

Adhesive layers of the lip-shaped elements 40A and 40B are adhered to the release sheet 44, and a user removes the lip-shaped elements 40A and 40B from the release sheet 44 prior to use. Several lip-shaped elements having different shapes may be attached to the release sheet 44 such that the user can select lip-shaped elements that most closely resembles shapes of the user's lips.

FIG. 4 is a cross sectional view of the cosmetic system 38 of FIG. 3 as indicated in FIG. 3, wherein the lip-shaped elements 40A and 40B are releasably attached to the release sheet 44. The lip-shaped elements 40A and 40B are mounted on the release sheet 44 via the adhesive layers so that they can be easily removed and applied. As illustrated in FIG. 4, the lip-shaped element 40A includes a body 46A. The body 46A includes a laminate structure having a thin film substrate 30A with an adhesive layer 32A formed on one side and a coloring layer 34A formed on an opposite side. The lip-shaped element 40B includes a body 46B. The body 46A includes a laminate structure having a thin film substrate 30B with an adhesive layer 32B formed on one side and a coloring layer 34B formed on the other side. The substrates 30A and 30B, the adhesive layers 32A and 32B, and the coloring layers 34A and 34B are similar to the respective substrate 30, adhesive layers 32, and coloring layer 34 of FIG. 2.

In general, the bodies 46A and 46B of the respective lip-shaped elements 40A and 40B may be formed from any suitable non-toxic materials. In a preferred embodiment, the bodies 46A and 46B also include a sugar or other sweet-tasting material.

In the embodiment of FIG. 4, the body 46A of the lip-shaped element 40A has a thicker center portion 52A and thinner top and bottom portions 50A and 54A, respectively, such that when the lip-shaped element 40A is attached to a person's lip, the person's lip appears full and plump. Similarly, the body 46B of the lip-shaped element 40B has a thicker center portion 52B and thinner respective top and bottom portions 50B and 54B for the same reason.

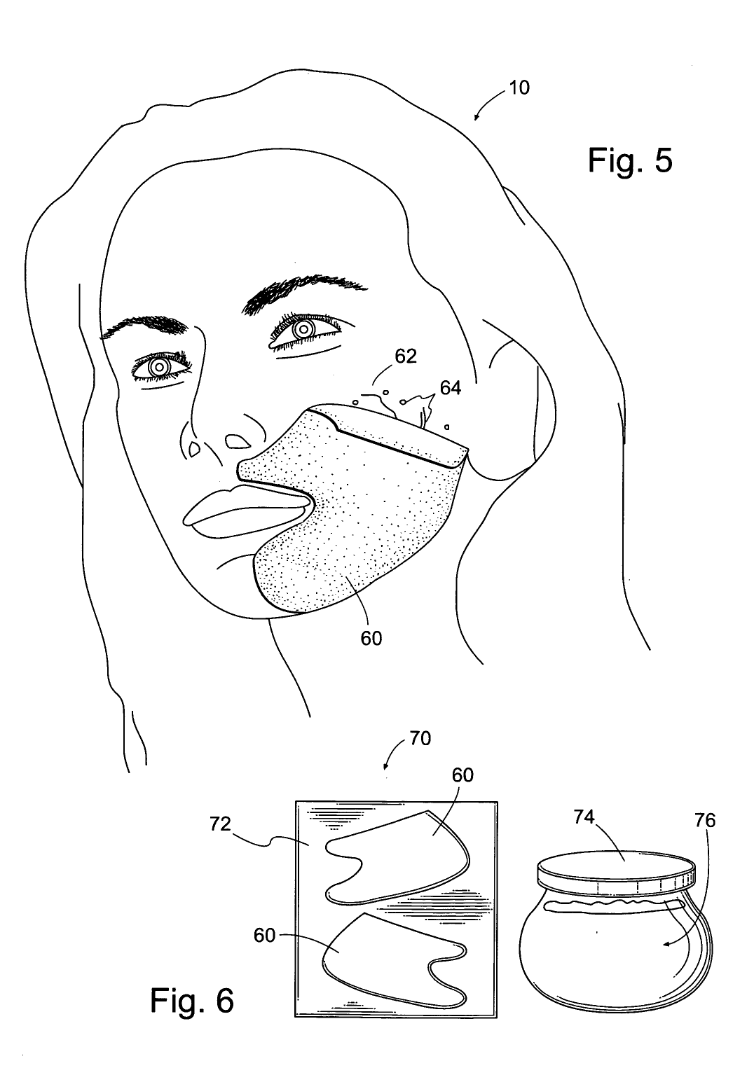

FIG. 5 is a perspective view of a third embodiment of the cosmetic device 10 of FIG. 1, wherein the cosmetic device 10 is a facial sheet 60 adapted to be applied to a person's face 62. The facial sheet 60 is preferably relatively thin, flexible, and adheres to the skin of the face 62. The facial sheet 60 is preferably thin enough to avoid being visually detectable, but also thick enough to cover imperfections 64 on the face 62 such as blemishes, acne scaring, etc. In general, the facial sheet 60 has a smooth outer surface that hides the existence of the imperfections 64.

The facial sheet 60 includes the laminate structure of FIG. 2 having the thin film substrate 30 with the adhesive layer 32 formed on one side and the coloring layer 34 formed on the opposite side. As described above, the coloring layer 34 includes a coloring agent to simulate either a makeup color or a human skin color. Further, the coloring layer 34 preferably has an outer surface that reflects light in substantially the same way that human skin reflects light.

FIG. 6 is a view of a cosmetic system 70 including two copies of the facial sheet 60 of FIG. 5 releasably mounted to a release sheet 72 and a container 74 containing a concealer 76. The two copies of the facial sheet 60 are mounted on the release sheet 72 via the adhesive layers so that they can be easily removed and applied.

In general, the concealer 76 is a facial cosmetic used to cover blemished skin. Suitable viscous liquid concealers are well known and widely available. The facial sheet 60 of FIGS. 5 and 6 is preferably used with the concealer 76. Once the facial sheet 60 has been applied to cover the imperfections 64 of FIG. 5, the concealer 76 is applied around the edges of and/or over the facial sheet 60 to hide the existence of the facial sheet 60.

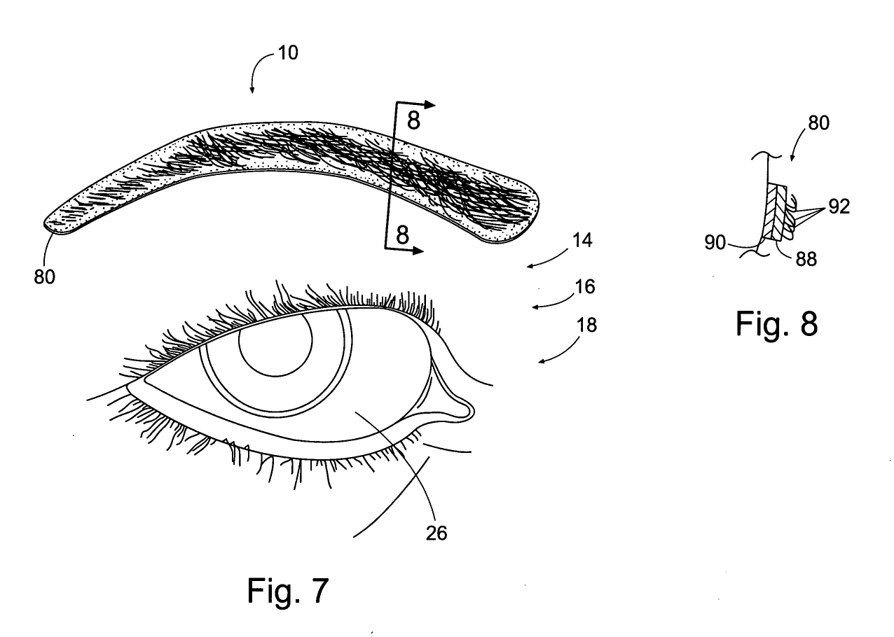

FIG. 7 is a perspective view of another embodiment of the cosmetic device 10 of FIG. 1, wherein the cosmetic device 10 is an eyebrow element 80 adapted to resemble a human eyebrow. The eyebrow element 80 may be used to replace a missing eyebrow, or to cover up an unattractive or imperfect eyebrow. In FIG. 7, objects shown in FIG. 1 and described above are numbered similarly. The eyebrow element 80 is expectedly positioned in the normal eyebrow position over a person's eye as shown in FIG. 7. More specifically, the eyebrow element 80 is expectedly positioned just above the upper portion 14 of the upper eyelid 16 of the person's face 18.

FIG. 8 is a cross sectional view of one embodiment of the eyebrow element 80 of FIG. 7 as indicated in FIG. 7. In the embodiment of FIG. 8, the eyebrow element 80 is a laminate structure including a substrate 88 with an adhesive layer 90 formed on one side. In general, the substrate 88 is formed from a thin, flexible material. The substrate 88 may be, for example, formed from a thin plastic film material. In general, the material used to form the substrate 88 is preferably not be readily visible on the skin. The material used to form the substrate 88 may be, for example, substantially transparent, or may include a coloring agent that simulates a makeup color or a human skin color. The substrate 88 preferably has an outer surface that reflects light in substantially the same way that human skin reflects light. Suitable plastic materials for the substrate 88 are believed to include polyethylene, polypropylene, polyvinyl chloride, and polyurethane. The adhesive layer 90 may be similar to the adhesive layer 32 shown in FIG. 2 and described above.

To reduce skin irritation, the substrate 88 preferably has many small holes passing therethrough (i.e., is preferably porous), and the adhesive layer 90 preferably conforms to the porous pattern of the substrate 30 such that the underlying skin can “breathe.” For example, the substrate 88 may be formed from a thin film material having many small holes (i.e., from a “microporous” thin film material).

In the embodiment of FIG. 8, multiple hair elements 92 are distributed across a surface of a side of the substrate 88 opposite the adhesive layer 90, and portions of the hair elements 92 extend outwardly from the side of the substrate 88 opposite the adhesive layer 90. In general, the hair elements 92 are adapted to resemble human eyebrow hairs. More specifically, the hair elements 92 preferably have lengths, thicknesses, and colors to simulate human eyebrow hairs, and are preferably curved like human eyebrow hairs.

In the embodiment of FIG. 8, each of the hair elements 92 has two opposed ends. One end of each of the hair elements 92 is anchored in the substrate 88, and the opposite end is free. In other embodiments both ends of the hair elements 92 may be anchored in the 88. Alternately, one or both ends of each of the hair elements 92 may be attached to the surface of the side of the substrate 88 opposite the adhesive layer 90.

It is noted that the adhesive layer of the cosmetic device 10 of FIGS. 1-8 allows the cosmetic device 10 to be attached to facial skin, and to be removed from the skin without damaging the skin. The different embodiments of the cosmetic device 10 may be used to give the appearance of professionally applied eye-shadow (FIGS. 1-2), to enhance a person's lips (FIGS. 3-4), to cover facial imperfections (FIGS. 5-6), or to replace a missing eyebrow or to cover up an unattractive or imperfect eyebrow (FIGS. 7-8).

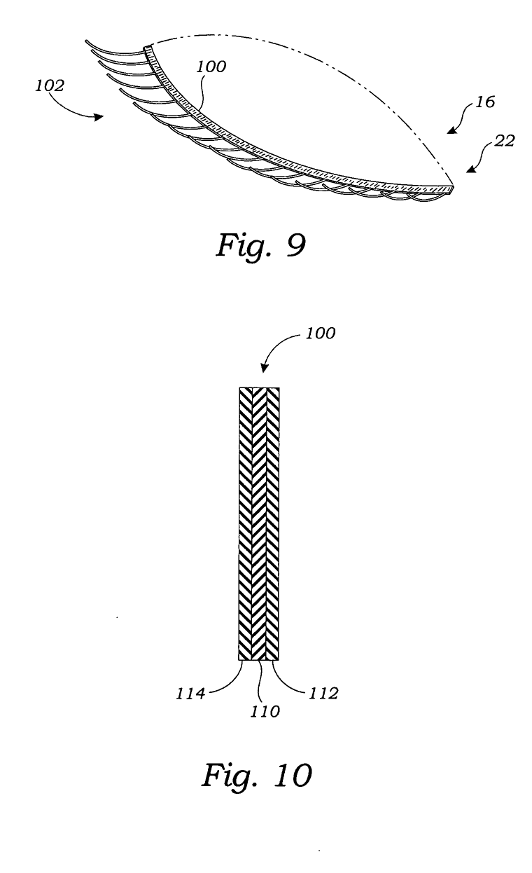

FIG. 9 is a perspective view of an eyeliner element 100 applied to the lower portion 22 of the upper eyelid 16 (see FIG. 1) just above an upper eyelash 102. As shown in FIG. 9, the eyeliner element 100 includes an elongate body (element number 110 in FIG. 10) that is long and thin. In the embodiment of FIG. 9, the eyeliner element 100 is flexible and preferably dark in color. When applied as shown in FIG. 9, the eyeliner element 100 resembles eyeliner makeup applied by hand—a dark line used to emphasize the contour of the lower portion 22 of the upper eyelid 16. As described in more detail below, the eyeliner element 100 includes an adhesive that sticks to the skin on contact.

While the eyeliner element 100 is applied to the lower portion 22 of the upper eyelid 16 just above an upper eyelash 102 in FIG. 9, it is noted that the eyeliner element 100 may also be applied to an upper portion of a lower eyelid just below a lower eyelash to emphasize the contour of the upper portion of the lower eyelid.

FIG. 10 is a cross sectional view of one embodiment of the eyeliner element 100 of FIG. 9. In the embodiment of FIG. 10, the eyeliner element 100 is a laminate structure including an elongate body 110 formed of a thin film substrate with an optional adhesive layer 112 formed on one side and a coloring layer 114 formed on an opposite side. In general, the substrate 110 is formed from a thin, flexible material. The substrate 110 may be formed from, for example, a thin plastic film material. Suitable plastic materials for the substrate 30 are believed to include polyethylene, polypropylene, polyvinyl chloride, and polyurethane.

The substrate 110 may have an adhesive quality such that the substrate 110 will adhere to human skin on contact, and can be removed from the skin without damaging the skin. In this situation, the optional adhesive layer 112 may not be required. For example, as described above, GLAD® Press 'n Seal™ sealable plastic wrap is a thin plastic film that either includes an adhesive layer or is inherently adhesive, and may be a suitable material from which to form the substrate 110.

The optional adhesive layer 112, when included, is preferably a layer of a pressure sensitive adhesive material that adheres to human skin on contact, and can be removed from the skin without damaging the skin. The adhesive material is preferably non-toxic and non-irritating. Suitable pressure sensitive adhesive material are well known in the medical and cosmetic arts, and are believed to include acrylic adhesives, rubber based adhesives, and styrene adhesives. The optional layer 112 may also be a layer of a water based and/or water washable adhesive material.

To reduce skin irritation, the substrate 110 preferably has many small holes passing therethrough (i.e., is preferably porous). When included, the optional adhesive layer 112 preferably conforms to the porous pattern of the substrate 110 such that the underlying skin can “breathe.” For example, the substrate 110 may be formed from a thin film material having many small holes (i.e., from a “microporous” thin film material).

In general, the coloring layer 114 includes a coloring agent that simulates either a makeup color. The coloring agent may be, for example, a suitable paint or a stain, and is preferably water resistant. The coloring agent may be, for example, sprayed (e.g., airbrushed) onto the substrate 110.

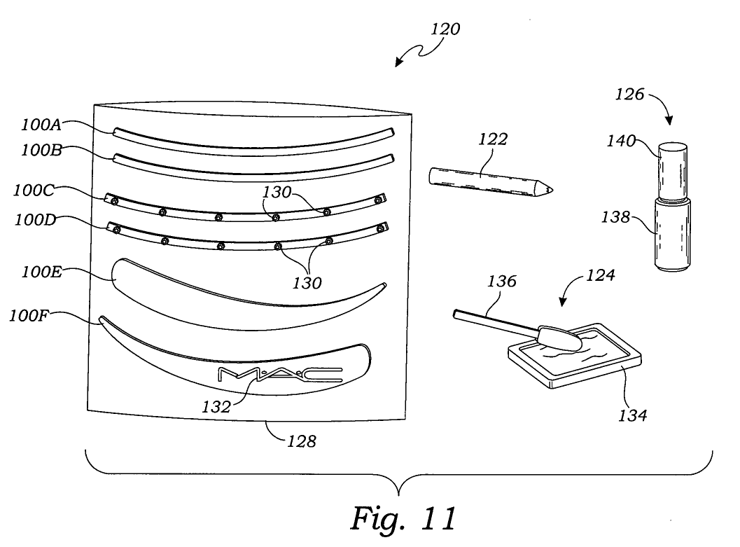

FIG. 11 is a view of one embodiment of a cosmetic system 120 including various embodiments of pairs of the eyeliner element 100 of FIG. 9, an eyeliner pencil 122, an eye shadow system 124, and a mascara system 126. In FIG. 11 the pairs of the eyeliner element 100 are releasably attached to a release sheet 128. A first of the pairs of the eyeliner element 100 includes an eyeliner element 100A and an eyeliner element 100B. A second of the pairs of the eyeliner element 100 includes an eyeliner element 100C and an eyeliner element 100D, and a third of the pairs of the eyeliner element 100 includes an eyeliner element 100E and an eyeliner element 100F.

As shown in FIG. 11, the eyeliner elements 100C and 100D include ornamental stones such as rhinestones 130 attached to outer surfaces along their lengths. The ornamental stones may also include any similar or equivalent jewels, jems, precious or semi-precious stones, or any other similar decorative feature. In the embodiment of FIG. 11, the rhinestones 130 are attached to the outer surfaces of the eyeliner elements 100C and 100D at regular intervals along their lengths.

As shown in FIG. 11, the corresponding eyeliner elements 100E and 100F are wider than the other eyeliner elements, and the eyeliner element 100F has a logo 132 formed on an outer surface. In general, the logo 132 is string of characters, a sign, or a symbol. For example, the logo 132 may be a name of a company or a trademark.

In the embodiment of FIG. 11, the eye shadow system 124 includes a block 134 of dry eye shadow and an applicator 136. The mascara system 126 includes a container 138 containing a viscous liquid makeup. The container 138 includes a cap 140. An applicator is attached to an underside of the cap 140 such that when the cap 140 seals an opening in the container 138, the applicator is located within the container 138.

In general, the eyeliner elements 100A-100F, the eyeliner pencil 122, the eye shadow system 124, and the mascara system 126 are color coordinated. For example, the eyeliner elements 100A-100F, the eyeliner pencil 122, the eye shadow system 124, and the mascara system 126 may be different shades of the same color. Alternately, the eyeliner elements 100A-100F, the eyeliner pencil 122, the eye shadow system 124, and the mascara system 126 may be different colors that form an attractive combination or “look.”

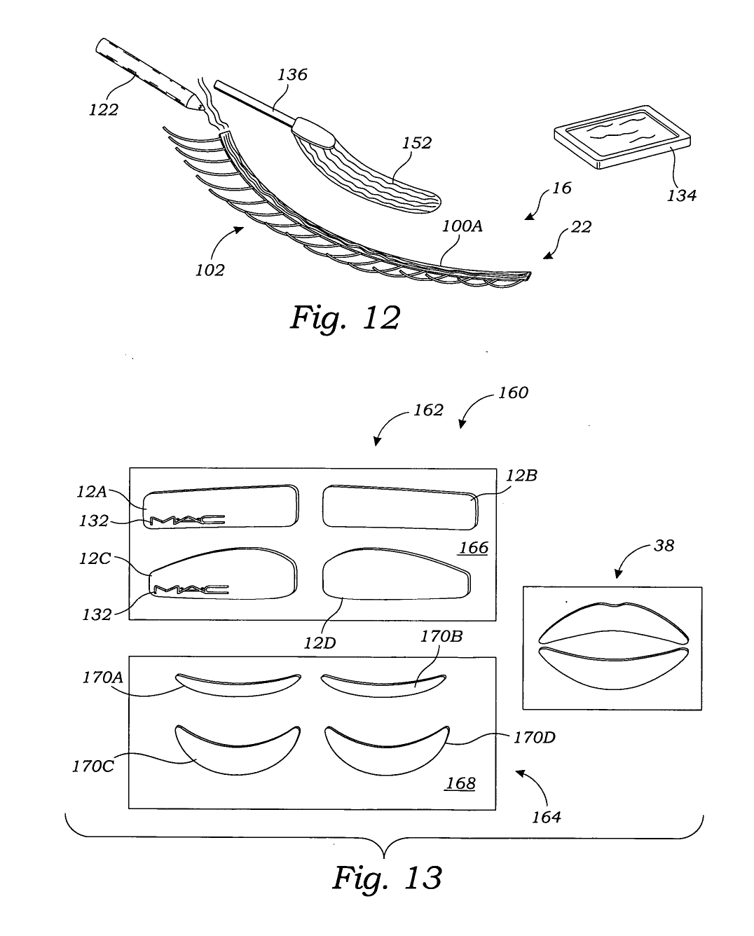

FIG. 12 is a perspective view of an eye 150 during application of components of the cosmetic system 120 of FIG. 11. In FIG. 12 the eyeliner element 100A has been applied to the lower portion 22 of the upper eyelid 16 just above the upper eyelash 102. The eyeliner pencil 122 is being used to extend a line or arc defined by the eyeliner element 100A beyond an end of the eyeliner element 100A. The applicator 136 of the eye shadow system 124 is being used to apply eye shadow 152 (obtained from the block 134 of dry eye shadow) to the upper eyelid 16.

FIG. 13 is a view of one embodiment of a cosmetic system 160 including a cosmetic system 162, a cosmetic system 164, and the cosmetic system 38 shown in FIGS. 3 and 4 and described above. The cosmetic system 162 includes two pairs of eye shadow elements 12 (see FIGS. 1 and 2). In the embodiment of FIG. 13 the pairs of the eye shadow elements 12 are releasably attached to a release sheet 166. A first pair of the eye shadow elements 12 includes an eye shadow element 12A and an eye shadow element 12B, and the second pair of the eye shadow elements 12 includes an eye shadow element 12C and an eye shadow element 12D.

As shown in FIG. 13, the eye shadow elements 12A and 12C have the logo 132 formed on an outer surface. As described above, the logo 132 is generally string of characters, a sign, or a symbol. For example, the logo 132 may be a name of a company or a trademark.

The cosmetic system 164 includes two pairs of under eye concealer elements releasably attached to a release sheet 168. A first of the pairs of the under eye concealer elements includes an under eye concealer element 170A and an under eye concealer element 170B. The second pair of the under eye concealer elements includes an under eye concealer element 170C and an under eye concealer element 170D.

In the embodiment of FIG. 13, the under eye concealer elements 170 are structured similarly to the eye shadow elements 12. That is, the under eye concealer elements 170 include a thin film substrate like the substrate 30 shown in FIG. 2 and described above, and a coloring layer like the coloring layer 34 shown in FIG. 2 and described above. In general, the under eye concealer elements 170 adhere to human skin on contact, and can be removed from the skin without damaging the skin. For example, the substrate may have an adhesive quality as described above. Alternately, the under eye concealer elements 170 may include an adhesive layer like the adhesive layer 32 shown in FIG. 2 and described above.

In general, the under eye concealer elements 170 are applied to an upper portion 172 of a lower eyelid 174 similar to the way the eye shadow elements 12 are applied to upper eyelids (see FIG. 1). When applied to lower eyelids, the under eye concealer elements 170 resemble eye shadow makeup applied by hand, or can be the color of human skin. Further, the structures of the under eye concealer elements 170 may be dimensioned to extend below the lower eyelids, and may help to conceal any wrinkles or “bags” under the eyes.

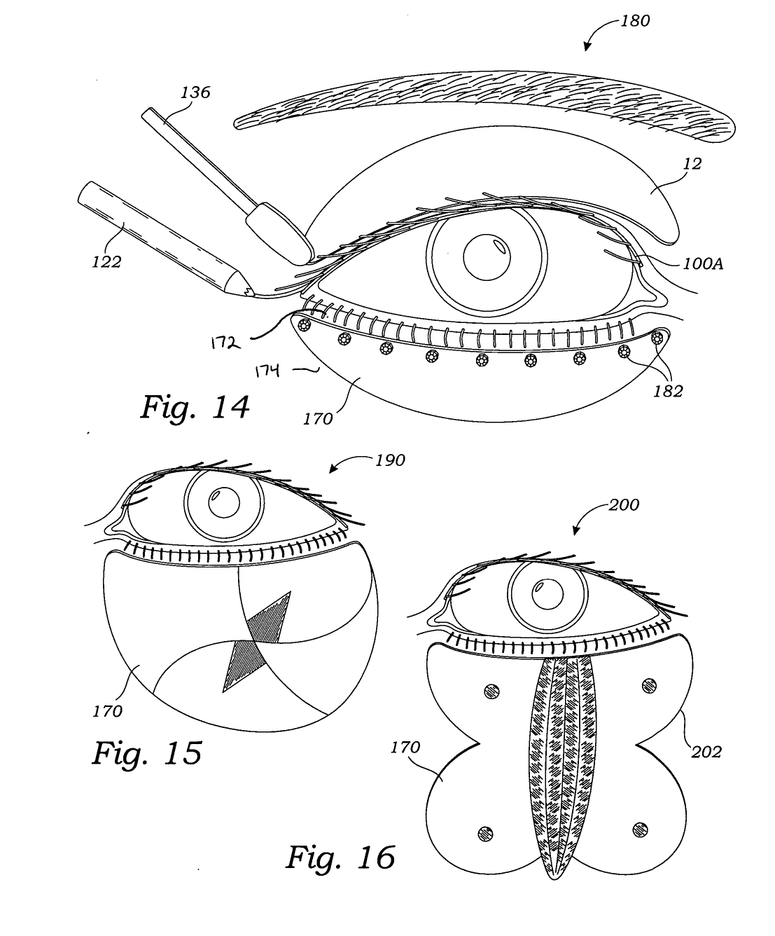

FIG. 14 is a perspective view of an eye 180 during application of components of the cosmetic system 120 of FIG. 11 and the cosmetic system 160 of FIG. 13. In FIG. 14 the eyeliner element 100A of the cosmetic system 120 of FIG. 11 has been applied to the lower portion of the upper eyelid just above the upper eyelash. An eye shadow element 12 of the cosmetic system 160 of FIG. 13 has been applied to the upper portion of the upper eyelid, and an under eye concealer element 170 of the cosmetic system 160 has been applied to the lower eyelid. The eyeliner pencil 122 of the cosmetic system 120 is being used to extend a line or arc defined by the eyeliner element 100A beyond an end of the eyeliner element 100A. The applicator 136 of the eye shadow system 124 of the cosmetic system 120 is being used to apply eye shadow to the upper eyelid.

In the embodiment of FIG. 14, the under eye concealer element 170 has rhinestones 182 attached to an outer surface along an upper edge. More specifically, the rhinestones 182 are attached to the outer surface of the under eye concealer element 170 at regular intervals along the upper edge.

FIG. 15 is a perspective view of an eye 190 wherein an under eye concealer element 170 has been applied to the lower eyelid. In the embodiment of FIG. 15, the under eye concealer element 170 extends below the lower eyelid and over a portion of an adjacent cheek, and includes several different colors forming a visual pattern or graphic.

FIG. 16 is a perspective view of an eye 200 wherein an under eye concealer element 170 has been applied to the lower eyelid. In the embodiment of FIG. 16, the under eye concealer element 170 extends below the lower eyelid and over a portion of an adjacent cheek, and forms an image. The under eye concealer element 170 has a perimeter 202 that substantially conforms to an outer edge of the image. More specifically, in the embodiment of FIG. 16, the under eye concealer element 170 forms an image of a butterfly, and the perimeter 202 substantially conforms to an outer edge of the image of the butterfly.

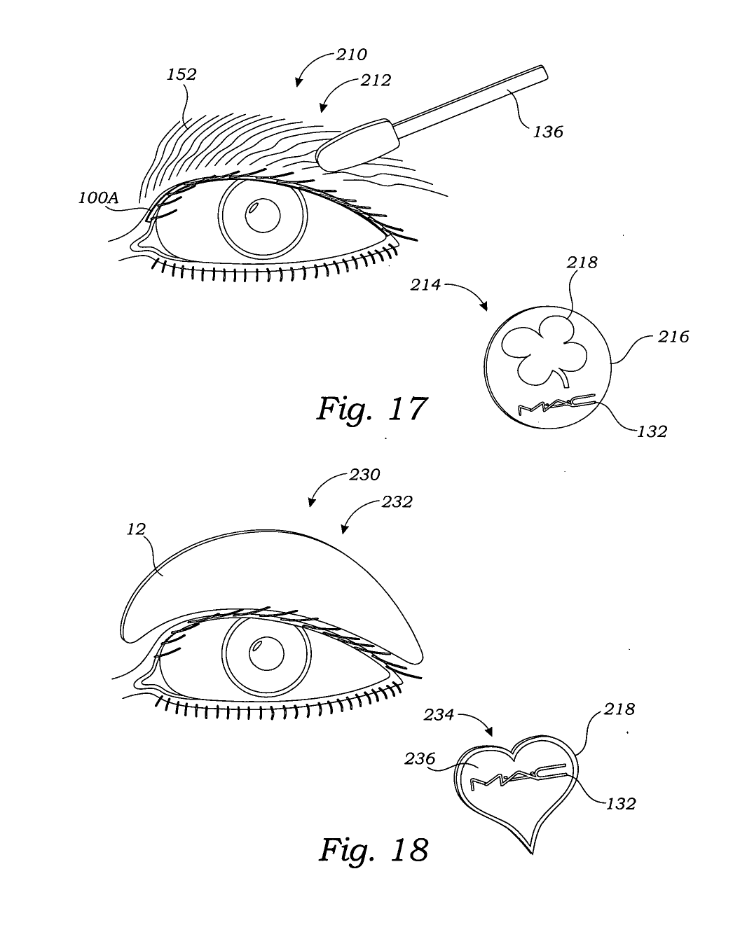

FIG. 17 is a perspective view of a portion of a face 210 including an eye 212 and an underlying cheek 214. In FIG. 17 the eyeliner element 100A of the cosmetic system 120 of FIG. 11 has been applied to the lower portion of the upper eyelid just above the upper eyelash, and the applicator 136 of the eye shadow system 124 of the cosmetic system 120 is being used to apply the eye shadow 152 to the upper eyelid.

In the embodiment of FIG. 17, a sticker 216 has been applied to the cheek 214. In general, the sticker 216 includes a substrate layer and a coloring layer on one side of the substrate. The sticker 216 adheres to human skin on contact, and can be removed from the skin without damaging the skin. For example, the substrate or the sticker 216 may have an adhesive quality as described above. Alternately, the sticker 216 may include an adhesive layer on a side of the substrate opposite the coloring layer.

In general, the sticker 216 includes several different colors forming a visual pattern or graphic. In the embodiment of FIG. 17, the sticker 216 includes an image 218 of a four-leaf clover. The sticker 216 also has the above described logo 132 formed on an outer surface.

In general, the colors of the sticker 216 are coordinated with colors of the eyeliner element 100A and the eye shadow 152. For example, the eyeliner element 100A may be a dark shade of green, the eye shadow 152 may be a light shade of green, and the image 218 of a four-leaf clover may be a medium shade of green. In combination, the eyeliner element 100A, the eye shadow 152, and the sticker 216 may form a “Saint Patrick's Day” look.

FIG. 18 is a perspective view of a portion of a face 230 including an eye 232 and an underlying cheek 234. In FIG. 18 an eye shadow element 12 of the cosmetic system 160 of FIG. 13 has been applied to the upper portion of the upper eyelid, and a sticker 236 has been applied to the cheek 234. In general, the sticker 236 includes a substrate layer and a coloring layer on one side of the substrate. The sticker 236 adheres to human skin on contact, and can be removed from the skin without damaging the skin. For example, the substrate or the sticker 236 may have an adhesive quality as described above. Alternately, the sticker 236 may include an adhesive layer on a side of the substrate opposite the coloring layer.

In general, the sticker 236 forms an image, and has a perimeter 238 that conforms to an outer edge of the image. In the embodiment of FIG. 18, the sticker 236 forms an image of a heart, and the perimeter 238 substantially conforms to an outer edge of the image of the heart. In FIG. 18 the sticker 236 also has the above described logo 132 formed on an outer surface.

In general, a color or colors of the sticker 236 are coordinated with a color or colors of the eye shadow element 12. For example, the eye shadow element 12 may be a dark shade of red, and the sticker 236 may include one or more different shades of red. In combination, the eye shadow element 12 and the sticker 236 may form a “Saint Valentine's Day” look.

While the invention has been described with reference to at least one preferred embodiment, it is to be clearly understood by those skilled in the art that the invention is not limited thereto. Rather, the scope of the invention is to be interpreted only in conjunction with the appended claims.

What is claimed is:

1. An eyeliner element adapted to be applied to a lower portion of an upper eyelid, the eyeliner element comprising:
an elongate body formed of a thin film substrate, the elongate body being shaped to fit adjacent the lower portion of the upper eyelid;
an adhesive layer formed on one side of the elongate body; and
a coloring layer formed on an opposite side of the elongate body.

2. The eyeliner element of claim 1, wherein the thin film substrate is a thin plastic film material.

3. The eyeliner element of claim 2, wherein the thin plastic film material includes polyethylene, polypropylene, polyvinyl chloride, and/or polyurethane.

4. The eyeliner element of claim 3, wherein the coloring layer is dark.

5. The eyeliner element of claim 1, wherein the elongate body includes ornamental stones attached thereto.

6. The eyeliner element of claim 5, wherein the ornamental stones are rhinestones.

7. An under eye concealer element adapted to be applied to an upper portion of a lower eyelid, the under eye concealer element comprising:
a thin film substrate shaped to fit adjacent the upper portion of the lower eyelid;
an adhesive layer formed on one side of the elongate body; and
a coloring layer formed on an opposite side of the elongate body.

8. The under eye concealer element of claim 7, wherein the thin film substrate is a thin plastic film material.

9. The under eye concealer element of claim 8, wherein the thin plastic film material includes polyethylene, polypropylene, polyvinyl chloride, and/or polyurethane.

10. The under eye concealer element of claim 7, wherein the elongate body includes ornamental stones attached thereto.

11. The under eye concealer element of claim 10, wherein the ornamental stones are rhinestones.
